(19) United States
(12) Patent Application Publication
(10) Pub. No.: US 2026/0294627 A1
(43) Pub. Date: Oct. 1, 2026

(54) SYSTEMS AND METHODS FOR TARGET/ARTICLE BRIDGING ENGAGEMENT AND ANCHORING

(71) Applicant: Valcare Medical, Inc., Wilmington, DE (US)

(72) Inventors: Aviad Galler, Tel Aviv (IL); Nadav Yellin, Even Yehuda (IL); Yoav Rozen, Binyamina (IL); Shuki Porath, Haifa (IL)

(73) Assignee: Valcare Medical, Inc., Wilmington, DE (US)

(21) Appl. No.: 19/698,676

(22) Filed: Jun. 4, 2026

Related U.S. Application Data
(63) Continuation of application No. 17/817,791, filed on Aug. 5, 2022, now Pat. No. 12,648,850.
(60) Provisional application No. 63/260,085, filed on Aug. 9, 2021.

(51) Int. Cl. A61F 2/24

(52) U.S. Cl. CPC A61F 2/2466; A61F 2/2445; A61F 2220/0016

(57) ABSTRACT

A system for bridging a gap between an article or a portion thereof and a target, and anchoring the article or its portion to the target, the system comprising a) an elongated delivery catheter having a distal end; b) and a bridging and anchoring construction, operable to selectably translate in a distal and a proximal direction relative to the distal end of the elongated delivery catheter, the bridging and anchoring construction slidably coupled to the elongated delivery catheter, wherein the bridging and anchoring construction is operable to 1) engage the target following the distal translation spanning the gap between the article or its portion and the target; 2) eliminating the gap following the proximal translation; and 3) anchoring the article or its portion to the target.

### CROSS-REFERENCE TO RELATED APPLICATIONS

This application is a continuation of U.S. patent application Ser. No. 17/817,791, filed Aug. 5, 2022, now U.S. Pat. No. 12,648,850, which claims priority to U.S. Provisional Application No. 63/260,085, filed Aug. 9, 2021, the entire contents of each of which are incorporated herein by reference.

### BACKGROUND

The present disclosure is directed to system and methods for bridging a gap between an article or a portion thereof and a target, and anchoring the article or its portion to the target. Specifically, the disclosure is directed to methods and systems for use in bridging the gap between an article or a portion thereof and a target, engaging the target, and anchoring the article or its portion to the target.

There are various circumstances in the medical field where bridging the gap between an implanted article and a target may be required and/or desired, as a stand-alone procedure, or as a part of other procedures, not necessarily as a method of treatment of underlying pathological condition.

Moreover, the ability to bridge the gap between the particular article and the target may be desirably done by minimally invasive means, such as, for example, trans-apical and trans-septal procedures to reduce post procedure recovery time and complications.

An example for such a need is in the process of repairing structural heart valves, such as the mitral valve, the tricuspid valve, the aortic valve and the pulmonary valves before, during or after annuloplasty ring is put into place.

The proposed technology aims to address the shortcomings of the current systems and methods.

### SUMMARY

In an exemplary implementation, provided herein is a system for bridging a gap between an article or a portion thereof and a target, and anchoring the article or its portion to the target, the system comprising: an elongated delivery catheter having a distal end; and a bridging and anchoring construction, operable to selectably translate in a distal and a proximal direction relative to the distal end of the elongated delivery catheter, the bridging and anchoring construction slidably coupled to the elongated delivery catheter, wherein the bridging and anchoring construction is operable to engage the target following the distal translation spanning the gap between the article or its portion and the target; eliminating the gap following the proximal translation; and anchoring the article or its portion to the target.

In another exemplary implementation, provided herein is a method for bridging a gap between an article or a portion thereof and a target, and anchoring the article or its portion to the target, the method implementable in a system comprising an elongated delivery catheter having a distal end; and a bridging and anchoring construction, operable to selectably translate in a distal and a proximal direction relative to the distal end of the elongated delivery catheter, the bridging and anchoring construction slidably coupled to the elongated delivery catheter, wherein the bridging and anchoring construction is operable to engage the target following a distal translation spanning the gap between the article or its portion and the target; eliminating the gap following a proximal translation; and anchoring the article or its portion to the target, the method comprising: inserting the delivery catheter; partially engaging the article or its portion; translating the bridging and anchoring construction in a distal direction relative to the delivery catheter's distal end over the gap; using the bridging and anchoring construction engaging the target; translating the bridging and anchoring construction in a proximal direction relative to the delivery catheter's distal end to the point where the target abuts the article or its portion; and using the bridging and anchoring construction anchoring the article or its portion to the target.

In yet another embodiment, provided herein is a use of a gap bridging and anchoring system in the process of bridging the gap between an article or a portion thereof and a target, and anchoring the article or its portion to the target, the system comprising: an elongated delivery catheter having a distal end; and a bridging and anchoring construction, operable to selectably translate in a distal and a proximal direction relative to the distal end of the elongated delivery catheter, the bridging and anchoring construction slidably coupled to the elongated delivery catheter, wherein the bridging and anchoring construction is operable to engage the target following a distal translation spanning the gap between the article or its portion and the target; eliminating the gap following a proximal translation; and anchoring the article or its portion to the target.

### BRIEF DESCRIPTION OF THE DRAWINGS

The method implementable using the gap bridging, engagement and anchoring systems disclosed herein will become apparent from the following detailed description when read in conjunction with the figures, which are exemplary, not limiting, and in which:

FIG. 1A is a schematic of a delivery catheter's distal end, FIG. 1B illustrating the delivery catheter in engagement configuration, with FIG. 1C illustrating enlarged section of the distal end of the delivery catheter illustrated in FIG. 1B, with FIG. 1D illustrating a Y-Z cross section of the delivery catheter illustrated in FIG. 1A, and FIG. 1E illustrating another schematic of a delivery catheter's distal end;

FIG. 2A, illustrating a first exemplary implementation of the bridging and anchoring construction, post engagement of the piercing tip, with FIG. 2B, illustrating an exploded view thereof, and FIG. 2C illustrating unstrained foreshaft in an anchoring configuration;

FIG. 3A, illustrating a second exemplary implementation of the bridging and anchoring construction, post engagement of the piercing tip, with FIG. 3B, illustrating an exploded view thereof, FIG. 3C illustrating the enlarged portion marked 3C in FIG. 3A, with FIG. 3D illustrating a Y-Z cross section of FIG. 3C along line B-B;

FIG. 4A, illustrating a third exemplary implementation of the bridging and anchoring construction, post engagement of the piercing tip, with FIG. 4B, illustrating an exploded view thereof, with unstrained foreshaft in an anchoring configuration;

FIG. 5A illustrates an exemplary implementation of the bridging and anchoring construction, with FIG. 5B, illustrating a X-Z cross section thereof;

FIG. 6A, illustrating an anchored annuloplasty ring anchored to a structural heart valve; with FIG. 6B, illustrating an exemplary implementation using more than one type of anchor;

FIG. 7A illustrates another exemplary implementation of the delivery catheter, operable to deliver the bridging and anchoring implementation as illustrated in FIGS. 7B-7E;

FIGS. 8A-8E, illustrates yet another exemplary implementation of the methods and systems disclosed; and

FIGS. 9A-9G, illustrate the use of delivery catheter as illustrated in FIG. 1E, to deliver the bridging and anchoring constructs disclosed.

While the disclosure of the system for bridging a gap between an article or a portion thereof and a target, and anchoring the article or its portion to the target, is amenable to various modifications and alternative forms, specifics thereof have been shown by way of example in the drawings and will be further described in detail below. It should be understood, however, that the intention is not to limit the disclosure to the particular exemplary implementations illustrated. On the contrary, the intention is to cover all obvious modifications, equivalents, and alternatives.

### DETAILED DESCRIPTION

Provided herein are exemplary implementations of methods and systems for use in bridging the gap between an article or a portion thereof and a target, engaging the target, and anchoring the article or its portion to the target. The target can be, for example: a pulmonary valve, a mitral valve, a tricuspid valve, an aortic valve and like valves and annuli requiring repair by implanting support structures. Similarly, the article can be an annuloplasty ring or a portion thereof, adapted, sized and configured to be anchored to the target.

Furthermore, in the context of the disclosure, the term ‘article, or its’ portion (whether toroidal or not), does not necessarily mean a ring defining a single plane, but rather encompasses other planes, for example a saddle-shaped ring. Likewise, the term “its' (first, second) portion” means, in the context of the disclosure, any part of the article operable to be adjoined to form the full article (e.g., annuloplasty ring).

### Definitions

The term “coupled”, including its various forms such as “operably coupling”, “coupling” or “couplable”, refers to and comprises any direct or indirect, structural coupling, connection or attachment, or adaptation or capability for such a direct or indirect structural or operational coupling, connection or attachment, including integrally formed components and components which are coupled via or through another component or by the forming process. Indirect coupling may involve coupling through an intermediary member or adhesive, or abutting and otherwise resting against, whether frictionally or by separate means without any physical connection.

In addition, for the purposes of the present disclosure, directional or positional terms such as “top”, “bottom”, “upper,” “lower,” “side,” “front,” “frontal,” “forward,” “rear,” “anterior”, “posterior”, “proximal”, “distal”, “rearward,” “back,” “trailing,” “leading,” “above,” “below,” “left,” “right,” “radial,” “vertical,” “upward,” “downward,” “outer,” “inner,” “exterior,” “interior,” “intermediate,”, “apical”, “basal”, etc., are merely used for convenience in describing the various exemplary implementations of the present disclosure and provide with an orientation of the various elements.

Likewise, the term “engage” and various forms thereof, when used with reference to an engaging element, for example in the engagement of proximal section 2012 of foreshaft 2016 with distal section 2021 of pushtube 202, refers in an exemplary implementation to the application of any forces that tend to hold proximal section 2012 and distal section 2021 together against inadvertent or undesired separating forces (e.g., such as may be introduced during manipulation of pushrod 202). It is to be understood, however, that engagement does not in all cases require the interlocking connection that is maintained against every conceivable type or magnitude of separating force. Further, the term “engaging element” refers in another exemplary implementation to one or a plurality of coupled components, at least one of which is configured for releasably engaging another element. Thus, this term encompasses both single part engaging elements and multi-part-assemblies, such as bridging and anchoring construction 20, 30, 40.

The terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., lance-member(s) 2014j includes one or more lance member).

Reference throughout the specification to “one exemplary implementation”, “another exemplary implementation”, “an exemplary implementation”, and so forth, means that a particular element (e.g., step, feature, structure, and/or characteristic) described in connection with the exemplary implementation is included in at least one exemplary implementation described herein, and may or may not be present in other exemplary implementations. In addition, it is to be understood that the described elements may be combined in any suitable manner in the various exemplary implementations.

In the context of the disclosure, the term “operable” means the system and/or the device, or a certain element or step is fully functional, sized, adapted and calibrated, comprises elements for, and meets applicable operability requirements to perform a recited function when activated, coupled, implemented, actuated, effected, or realized. In relation to systems, the term “operable” means the system is fully functional and calibrated, having the necessary elements, as well as the mechanisms for, and meets applicable operability requirements to perform a recited function when executed by a user.

The term “abut” refers in the context of the disclosure, to items that are in direct physical contact with each other, although the items may not be attached, secured, fused, glued, sewn, or welded together.

A more complete understanding of the methods and systems for use in bridging the gap between an article or a portion thereof and a target, engaging the target, and anchoring the article or its portion to the target, can be obtained by reference to the accompanying drawings. These figures (also referred to herein as “FIG.”) are merely schematic representations based on convenience and the ease of demonstrating the present disclosure, and are, therefore, not intended to indicate relative size, scale and dimensions of the devices or components thereof, and/or to define or limit the scope of the exemplary implementations. Although specific terms are used in the following description for the sake of clarity, these terms are intended to refer only to the particular structure of the exemplary implementations selected for illustration in the drawings, and are not intended to define or limit the scope of the disclosure. In the drawings and the following description below, it is to be understood that like numeric designations refer to components of like function.

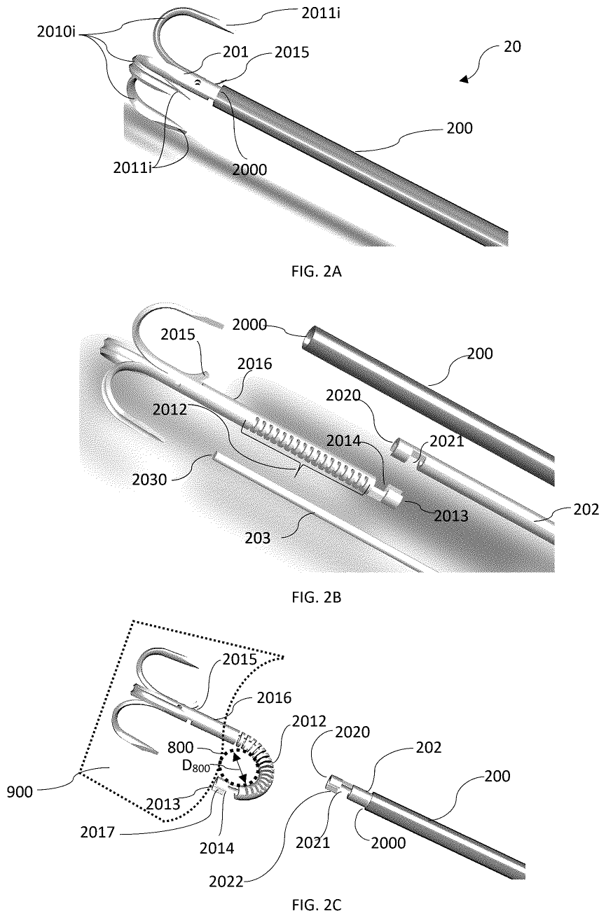

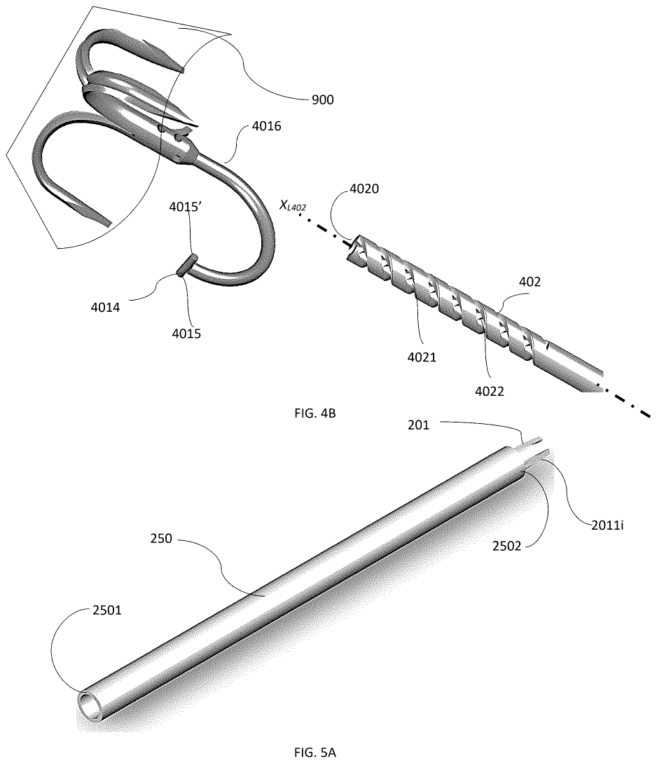

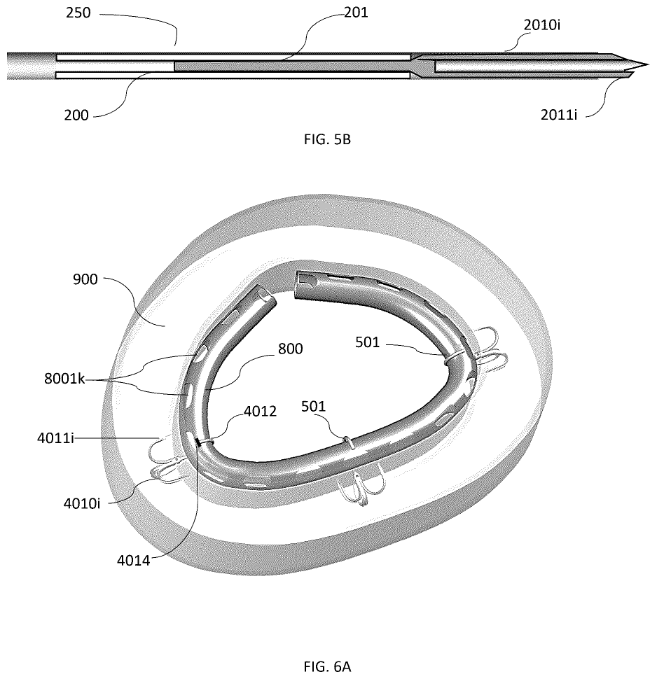

In the context of the disclosure, the term “strained” as used to describe the strained foreshaft (e.g., 201 see e.g., FIG. 2A) means that the foreshaft is maintained under an extrinsic tensile strain. An “extrinsic strain” as used herein refers to a tensile strain that is applied to the foreshaft by the sheath (or an external force exerted by another member), rather than a tensile strain developed within the strained foreshaft. In other words, in the unstrained position (see e.g., FIG. 2C), the foreshaft, formed of a resilient material (e.g., nitinol, stainless steel and the like) curls, and the insertion into sheath 200, or jacket 250 (see e.g., FIG. 5B) causes the tensile stress to form the strain imposed on the foreshaft or piercing tip's piercing lances.

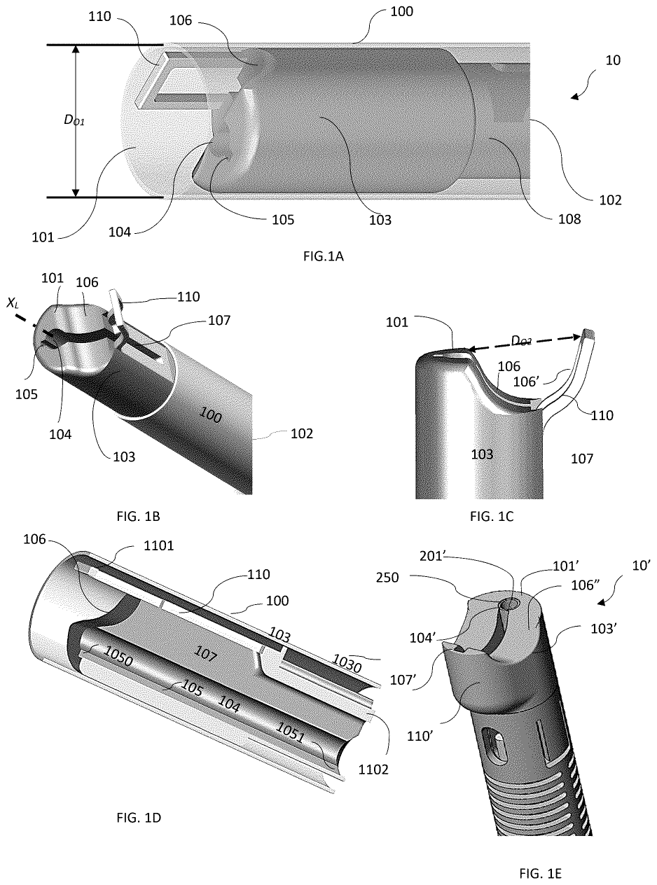

Turning now to FIGS. 1A-5B, illustrating exemplary implementation of the systems for use in bridging the gap between an article or a portion thereof and a target, engaging the target, and anchoring the article or its portion to the target. As illustrated, in FIG. 1A-2D, the system comprises: elongated delivery catheter 10, 10′ having distal end 101, 101′ (see e.g., FIG. s 1A, 1E); and bridging and anchoring construction (see e.g., 20, 30, 40 FIGS. 2A, 3A, 4A), each bridging and anchoring construction 20, 30, 40 operable to selectably translate in a distal and a proximal direction relative to distal end 101 of elongated delivery catheter 10, 10', each of bridging and anchoring construction 20, 30, 40 slidably coupled to elongated delivery catheter 10, 10′ wherein each bridging and anchoring construction 20, 30, 40 is operable to engage target 900 (see e.g., FIGS. 4B, 6A) following distal translation spanning the gap between article 800 (see e.g., FIG. s 2C, 6A, 6B) or its portion and target 900; eliminating the gap following proximal translation; and anchoring article 800 or its portion to the target 900.

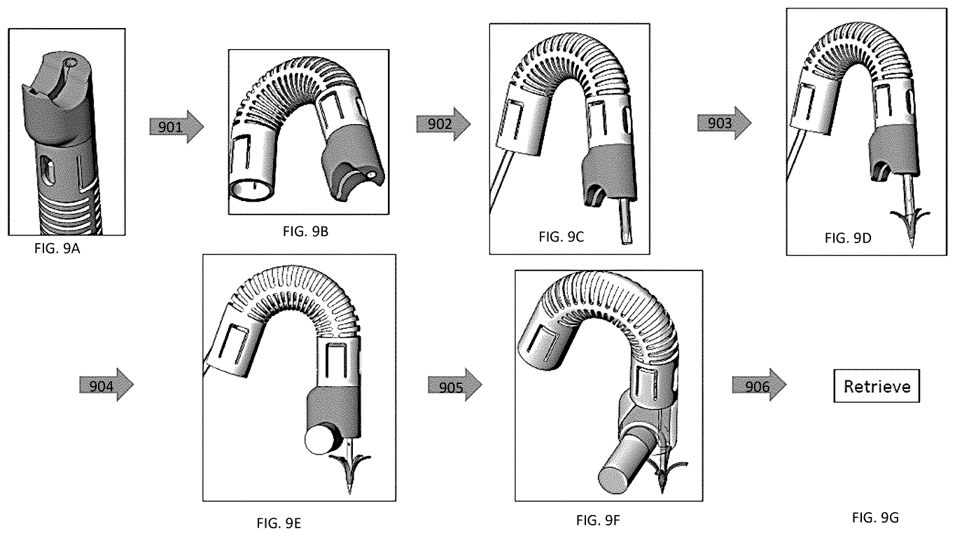

As further illustrated in FIG. 1A-1E, delivery catheter 10, is operable to transition between an insertion configuration as illustrated in FIG. s 1A, and 1D and engagement configuration illustrated e.g., in FIGS. 1B and 1C, wherein distal end 101 is contoured 106 to abut at least portion of article 800 or its portion's surface (see e.g., for elongated catheter 10′, FIG. 9F) in its engagement configuration (FIGS. 1B and 1C), elongated delivery catheter 10 further comprises: cylindrical sleeve 100, operable to slidably translate in proximal direction; cylindrical housing 103, defining through bore 104, sized to accommodate jacket 250 housing each of bridging and anchoring construction 20, 30, 40, cylindrical housing 103 having distal end 101 with arcuate cross section 106 (see e.g., FIG. 1D) sized to abut between about 1.0 radians, and about 4.5 radians of at least portion of article 800 (see e.g., FIGS. 2C, 9F) or its portion's surface. It is noted that surface 106 in distal end 101 of cylindrical housing 103 does not need to be specifically arcuate, but rather, in certain exemplary implementations, have a surface that is complementary to a predetermined portion of article 800 configured to be manipulated to assist in locating article 800 relative to target 900 (see e.g., FIG. 6A, and 9A-9F). Also illustrated in FIG. s 1A-1D, is expander 110, operable to transition between strained position within sleeve 100 and unstrained position outside sleeve 100, wherein, in its unstrained position (FIGS. 1B and 1C), distal portion 1101 (see e.g., FIG. 1D) of expander 110 forms complimentary arcuate cross section 106′ to arcuate cross section 106 of distal end 101 of cylindrical housing 103. Similarly, it is noted that surface 106, 106′ formed by distal end 101 of cylindrical housing 103, and expander 110 in its unstrained configuration does not need to be specifically arcuate, but rather form a surface that is complementary to a predetermined portion of article 800 configured to be manipulated to assist in locating article 800 relative to target 900.

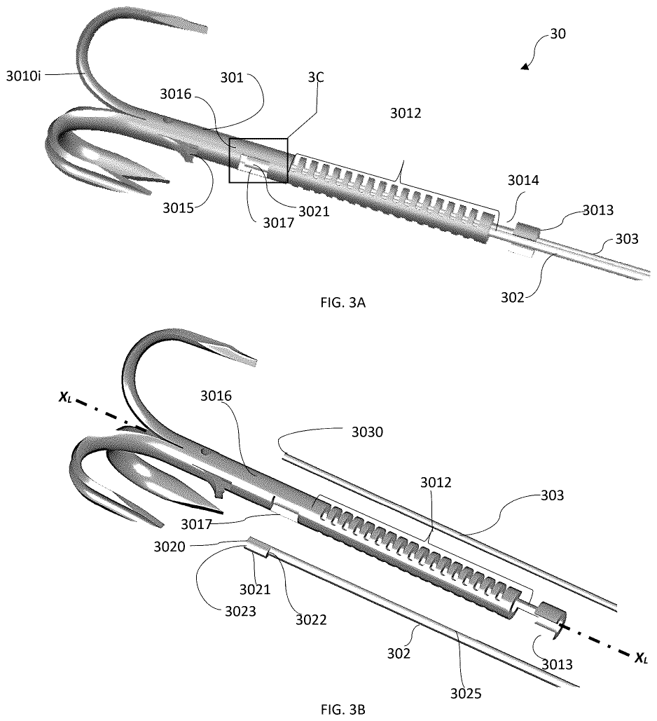

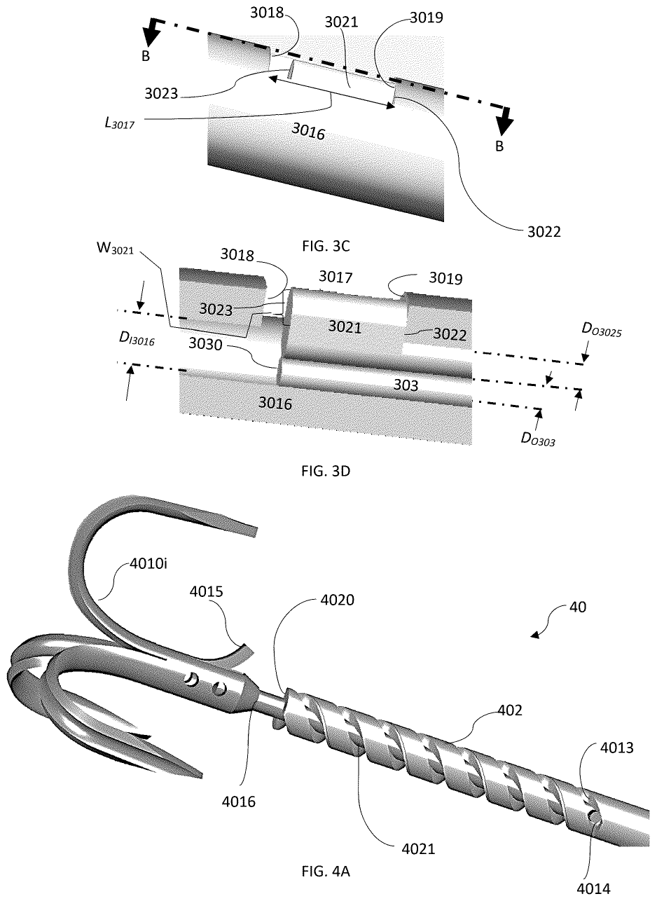

Also illustrated in FIGS. 1A, 1B, and 1D, is groove 105 configured to receive and accommodate each of protruding tab 2015, 3015, and 4015 of bridging and anchoring construction 20, 30, 40 respectively. Similarly, and as illustrated in FIG. 1B, slit 107 formed in cylindrical housing 103, is configured to accommodate each of foreshaft 201, 301, and 401 of bridging and anchoring construction 20, 30, 40 respectively is they transition from their strained to unstrained configuration as disclosed herein. As further illustrated in FIG. 1D, expander 110 having distal end 1101 and proximal end 1102, can be engaged, in an exemplary implementation, in channel 1030 within cylindrical housing 103 and be extrinsically strained by sleeve 100, such that upon proximal translation (pulling e.g.,) of sleeve I 00 beyond the interface of arcuate surface 106 with expander 110, expander 110 will transition to its unstrained configuration. Likewise, as illustrated in FIG. 1D, (applying also to the exemplary implementation illustrated in FIG. 1E) groove 105, having distal end 1050 and proximal end 1051, is configured to accommodate protruding tab 2015, 3015, and 4015 of bridging and anchoring construction 20, 30, 40 respectively thus preventing each of bridging and anchoring construction 20, 30, 40 from radially rotating about longitudinal axis X_{L} (see e.g., FIG. 3B) during the distal translation (pushing e.g.,) of each of bridging and anchoring construction 20, 30, 40.

In another exemplary implementation illustrated in FIG. 1E, elongated catheter 10′ comprises: cylindrical housing 103′, defining through bore 104′, sized to accommodate each of bridging and anchoring construction 20, 30, 40, (e.g., jacket 250 accommodating each anchoring construction 20, 30, 40), cylindrical housing 103′ having distal end 101′ with arcuate cross section 106′ sized to abut between about 1.0 radians, and about 4.5 radians of at least portion of article 800 (see e.g., FIGS. 2C, 9F) or its portion's surface. Also illustrated, is slit 107′ formed in cylindrical housing 103′, configured likewise to accommodate each of foreshaft 201, 301, and 401 of bridging and anchoring construction 20, 30, 40 respectively is they transition from their strained to unstrained configuration as disclosed herein (see e.g., FIG. 9F).

Turning now to FIGS. 2A-2C, illustrating first exemplary implementation of bridging and anchoring construction 20, following engagement of piercing tip 2010, with FIG. 2B, illustrating an exploded view thereof, and FIG. 2C illustrating unstrained foreshaft 201 in an anchoring configuration. As illustrated, bridging and anchoring construction 20 used in the methods disclosed for the uses described, can comprises in an exemplary implementation: jacket 250, (see e.g., FIGS. 5A, 5B), sheath 200; piercing tip 2010 comprised of plurality of lancing members 2010i, each i^{th} piercing tip (e.g., between 3 and 5) 2010i having sharpened tip end 2011i, configured to transition between strained position within jacket 250 and unstrained position outside jacket 250 upon distal translation of piercing tip 2010 outside of jacket 250. Also shown, is tubular (in other words, axially hollow) foreshaft 201 having proximal end 2016, tubular foreshaft 201 distally coupled to piercing tip 2010, whereby tubular foreshaft 201 comprises proximal region 2012 configured to transition between strained position (see e.g., FIG. 2A) when coupled to at least one of: coupling rod 203 inserted coaxially within tubular foreshaft 201, and sheath 200, and unstrained position (see e.g., FIG. 2C) following proximally translating at least one of (in other words, and/or): coupling rod 203 inserted coaxially within the tubular foreshaft, and sheath 200; proximal end 2013 forming portion of proximal region 2012, proximal end 2013 operable to form engagement section 2014 with T-line interrupted wall. In other words, the T-line interrupted wall has a T-shape cut into the wall with the leg extending from proximal end 2013 to some distance culminating with the cross bar. The T-line interrupted wall is adapted, sized and configured to receive and accommodate a complementary engagement portion (e.g., 2021). Thus, bridging and anchoring construction 20 further comprises pushtube 202, having proximal end 2020 operable to form engagement section 2021 with T-line interrupted wall, complementary to engagement section 2014 of tubular foreshaft 201, releasably coupled coaxially to proximal end 2013 of tubular foreshaft 201. Moreover, and as indicated coupling rod 203, when present, sized to be accommodated coaxially within pushtube 202, extending into tubular foreshaft 201 in distal direction beyond strained proximal region 2012, thus providing an alternative, or additional extrinsic strain on proximal region 2012, which can be slotted with slots distanced such that the unconstrained configuration will define a predetermined shape, for example, an arc having predetermined internal diameter D₈₀₀ as illustrated in FIG. 2C.

Another exemplary implementation of bridging and anchoring construction 30 is illustrated in FIGS. 3A-3D, where FIG. 3A, illustrates bridging and anchoring construction 30, post engagement of the piercing tip 3010, with FIG. 3B, illustrating an exploded view thereof, FIG. 3C illustrating the enlarged portion marked 3C in FIG. 3A, and FIG. 3D illustrating a Y-Z cross section of FIG. 3C along line B-B. As illustrated, bridging and anchoring construction 30 comprises: jacket 250 (see e.g., generally FIGS. 5A, 5B) optionally sheath 200 (see e.g., FIG. 2B); piercing tip 3010 comprised of plurality of lancing members 3010i each i^{th} lancing member 3010i configured to transition between strained position within jacket 250 (see e.g., FIG. 5B) and unstrained position outside jacket 250 (see e.g., FIG. 3A). Similar to bridging and anchoring construction 20, bridging and anchoring construction 30 also comprises tubular foreshaft 301 having proximal end 3013, tubular foreshaft 301 distally coupled to piercing tip 3010, comprising proximal region 3012 configured to transition between strained position when coupled to pushrod 302 inserted coaxially within tubular foreshaft 301 and at least one of: coupling rod 303, and optionally sheath 200 (see e.g., FIG. 5B), and unstrained position following decoupling of pushrod 302 and proximal translation of coupling rod 303, and/or sheath 200. Tubular foreshaft 301 further defining radial opening 3017, defined in tubular foreshaft's 301 wall, radial opening 3017 defined distal to proximal region 3012, having proximal end 3019 (see e.g., FIG. 3C) and distal end 3018, radial opening 3017 being sized to accommodate distal portion 3021 of pushrod 302. Pushrod 302, with distal portion 3021 extending radially from pushrod's 302 distal end, wherein distal portion 3021 forms tab extending radially from pushrod 302 to distance W (see e.g., FIG. 3D), that is larger than radius (D_{I3016}/2) of internal diameter D_{I3016} of tubular foreshaft 301 and smaller than internal diameter D_{I3016} of tubular foreshaft 301 and wherein proximal portion 3025 (see e.g., FIG. 3B) of pushrod 302 has outer diameter D_{O3025}, that is not larger than radius (D_{I3016}/2) of internal diameter D_{I3016} of tubular foreshaft 301 (see e.g., FIG. 3D). Also illustrated is coupling rod 303, which, when used in certain exemplary implementations, is sized to be accommodated coaxially within tubular foreshaft 301 extending in distal direction beyond the strained proximal region 3012. Coupling rod 302 is sized to have outer diameter D_{O303} that is not larger than the (D_{I3016}/2) of internal diameter D_{I3016} of tubular foreshaft 301.

Yet another exemplary implementation of bridging and anchoring construction 40 is illustrated in FIGS. 4A and 4B, illustrating a third exemplary implementation of the bridging and anchoring construction 40, post engagement of the piercing tip 4010 (see e.g., FIG. 6B), with FIGS. 4B, illustrating an exploded view thereof, with unstrained foreshaft 401 in an anchoring configuration (see e.g., FIG. 6A). As illustrated in FIG. 4A, bridging and anchoring construction comprises: jacket 250; optionally sheath 200 (see e.g., FIG. 2B); piercing tip 4010 comprised of plurality of lancing members 4010i, each i^{th} lancing member 4010i configured to transition between strained position within jacket 250 and unstrained position outside jacket 250. Also shown is pushtube 402, having distal portion 4021 defining helical wall channel 4022, helical wall channel 4022 (see e.g., FIG. 4B) having predetermined pitch (referring to the distance along longitudinal axis X_{L402} to complete a 360° turn) configured to engage proximal portion 4013 of foreshaft 401. As illustrated in FIG. 4A, foreshaft 401 having distal end 4016 coupled to piercing tip 4010, and proximal portion 4013 with proximal end 4014 with pair of diametrically opposed pegs 4015, 4015′ extending laterally from proximal end 4014 of foreshaft 401 wherein: pair of diametrically opposed pegs 4005, 4015′ extending laterally are sized and configured to be accommodated in helical wall channel 4022 of pushtube 402; and proximal portion 4013 is configured to transition between strained position when engaged in pushtube 402 (see e.g., FIG. 4A) and unstrained position (see e.g., FIG. 4B) outside pushtube 402.

As indicated, the systems disclosed used as described, implement in certain exemplary implementations the methods provided. Accordingly, and in an exemplary implementation illustrated schematically in FIGS. 9A-9G, provided herein is a method for bridging a gap between an article or a portion thereof and a target, and anchoring the article or its portion to the target, the method implementable in a system comprising elongated delivery catheter 10, 10′ having distal end 101 101′; and bridging and anchoring construction 20, 30, 40, operable to selectably translate in a distal and a proximal direction relative to distal end 101, 101′ of elongated delivery catheter 10, 10′ bridging and anchoring construction 20, 30, 40 slidably coupled to elongated delivery catheter 10, 10′ wherein bridging and anchoring construction 20, 30, 40 is operable to engage target 900 following distal translation spanning gap between article 800 or its portion and target 900 (see e.g., FIG. 6A); eliminating the gap following proximal translation; and anchoring article 800 or its portion to target 900 (see e.g., FIGS. 6A, 6B), whereby the method comprises: inserting delivery catheter 10, 10′; partially engaging article 800 (901, FIGS. 9A, 9B) or its portion to delivery catheter 10, 10′; translating bridging and anchoring construction 20, 30, 40 in distal direction (902, FIGS. 9B, 9C) relative to delivery catheter's 10, 10′ distal end 101, 101′ over the gap; using bridging and anchoring construction 20, 30, 40, engaging target 900 (903, FIGS. 9C, 9D); translating bridging and anchoring construction 20, 30, 40 in proximal direction (pulling, (904, FIGS. 9D, 9E) relative to delivery catheter's 10, 10′ distal end 101, 101′ to a point where target 900 abuts article 800 (see e.g., FIG. 2C) or its portion; and using bridging and anchoring construction 20, 30, 40, anchoring (905, FIGS. 9E, 9F) article 800 or its portion to target 900 (see e.g., FIGS. 6A, 6B).

In the methods disclosed, the gap between the article or its portion and the target, is between about 0.01 mm and about 40 mm. In other words, the gap can be minimal and the use of the systems disclosed can be to permanently or temporarily anchor the article (e.g., an annuloplasty ring, see e.g., FIG. 6A) to the target, (for example, a mitral valve annulus, a tricuspid valve annulus, or a pulmonary valve annulus).

Furthermore, in another exemplary implementation, the step of transitioning delivery catheter 10 from insertion configuration (see e.g., FIG. 1A) to engagement configuration (see e.g., FIG. 1C), comprises retracting sleeve 100 in a proximal direction, causing expander 110 to transition to its unstrained position as illustrated in FIGS. 1B, and 1C, where cylindrical housing 103 having distal end 101 with arcuate cross section 106 (see e.g., FIG. 1D) sized to abut between about 1.0 radians, and about 4.5 radians of at least portion of article 800 (see e.g., FIG. 2C) or its portion's surface and expander 110, in its unstrained position (FIGS. 1B and 1C), distal portion 1101 (see e.g., FIG. 1D) of expander 110 forms complimentary arcuate cross section 106′ to arcuate cross section 106 of distal end 101 of cylindrical housing 103.

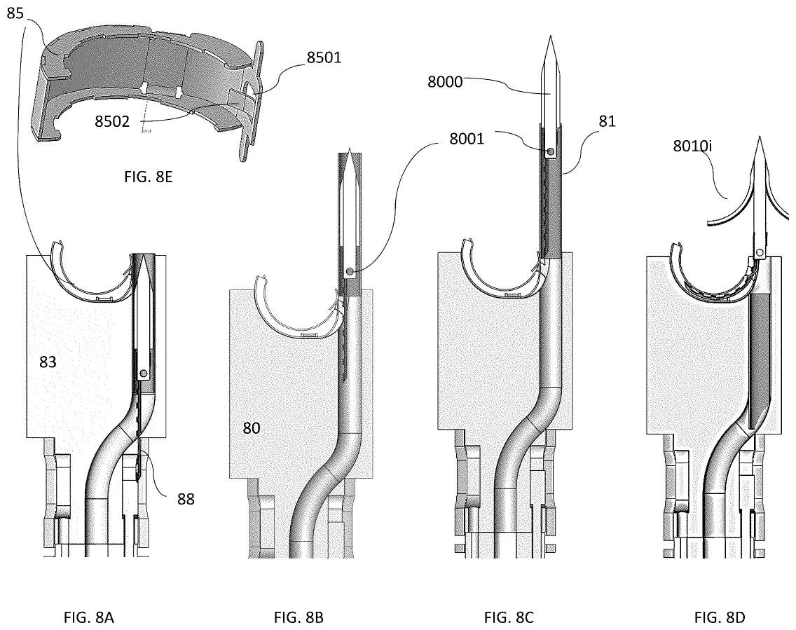

Thereafter, in an exemplary implementation, using bridging and anchoring construction 20, the step of translating bridging and anchoring construction 20 in a distal direction relative to delivery catheter's 10, 10′ distal end 101, 101′ further comprises: using pushtube 202, translating (see e.g., FIG. 9C) bridging and anchoring construction 20 in the distal direction; (see e.g., FIG. 8C) to the point where distal end 2502 (see e.g., FIG. 5A) of jacket 250 abuts target 900; using pushtube 202, distally translating piercing tip 2010 and piercing target 900 with piercing tip 2010. Moreover, the step of engaging target 900 comprises: distally translating piercing tip 2010 into target 900, causing each i^{th} lancing members 2010i to transition from a strained configuration inside jacket 250 to unstrained position outside jacket 250, reversing each i^{th} lancing members' 2010i direction toward the interior of target 900 toward the interface of target 900 and article 800 or its portion, thereby engaging target 900 (see e.g., FIG. 6A). In certain implementation, the transition of each i^{th} lancing members' 2010i itself, between the strained configuration inside jacket 250 to unstrained position outside jacket 250 in multiple direction within target 900 is the step causing the engagement between bridging and anchoring construction 20 lancing tip 2010 and target 900. Furthermore, the step of translating bridging and anchoring construction 20 in a proximal direction comprises: using pushtube, 202 translating bridging and anchoring construction 20 in a proximal direction, to the point where target 900 abuts article 800 or its portion (see e.g., FIG. 2C). Yet further, the step of anchoring article 800 or its portion to target 900 comprises: retracting sheath 200 in a proximal direction beyond distal end 2020 of pushtube 202; decoupling pushtube 202 from tubular foreshaft 201; and retracting coupling rod 203 beyond proximal end 2013 of tubular foreshaft 201, and/or retracting (in other words, translating in a proximal direction) sheath 200 beyond proximal end 2013 of tubular foreshaft 201, causing strained proximal region 2012 to transition to its unstrained configuration, hooking article 800 or its portion, thereby anchoring article 800 or its portion to target 900.

Thereafter, the step of translating bridging and anchoring construction 20 in a proximal direction (in other words, pulling the target toward the article) comprises: using pushtube 202, translating bridging and anchoring construction 20 in a proximal direction (pulling), to point where target 900 abuts article 800 or its portion; and the step of anchoring article 800 or its portion to target 900 comprises: retracting sheath 200 in a proximal direction beyond distal end 2020 of pushtube 202; disengaging pushtube 202 from tubular foreshaft 201 (e.g., by disengaging the interlocking T-line interrupted walls 2017 of engaging proximal portion 2014 of tubular foreshaft 201 and T-line interrupted wall 2022 of engaging distal portion 2021 of pushtube 202); and retracting coupling rod 203 beyond proximal end 2013 of tubular foreshaft 201, causing strained proximal region 2012 of tubular foreshaft 201 to transition to unstrained position, forming a hook over article 800 or its portion, thereby anchoring article 800 or its portion to target 900.

In a preliminary step, prior to the step of translating each of bridging and anchoring construction 20, 30, 40, and optionally following the step of transitioning delivery catheter 10 from insertion configuration (see e.g., FIG. 1A) to engagement configuration (see e.g., FIG. 1C), the method further comprises using delivery catheter 10, accommodating at least one bridging and anchoring construction 20, 30, 40, engaging article 800 to abut surface 106, 106′ formed by distal end 101 of cylindrical housing 103, and unstrained expander 110.

Additionally, or alternatively, in an exemplary implementation, of using bridging and anchoring construction 30, the step of translating bridging and anchoring construction 30 in a distal direction (in other words, pushing bridging and anchoring construction 30) relative to delivery catheter's 10, 10′ distal end 101, 101′ comprises: using pushrod 302, translating bridging and anchoring construction 30 in a distal direction (in other words, toward target 900) by pushing distal end 3023 of tab 3021 extending radially from pushrod 302 against distal end 3018 of radial opening 3017, defined in tubular foreshaft's 301 wall, to the point where distal end 2502 of jacket 250 abuts target 900,; distally translating piercing tip 3010 into target 900 thereby exposing piercing tip 3010; and piercing target 900 with piercing tip 3010. Consequently, the step of engaging target 900 comprises: further advancing jacket 250 in a distal direction, causing lancing members 3010i, once outside distal end 2502 of jacket 250 to transition to unstrained position outside jacket 250 reversing each i^{th} lancing members'3010i direction, thereby engaging target 900.

Thereafter, the step of translating bridging and anchoring construction 30 in a proximal direction (pulling target 900 toward article 800 or vice-a-versa) comprises: using pushrod 302, translating bridging and anchoring construction 30 in a proximal direction (toward the target) by pulling tab 3021 extending radially from pushrod 302 against proximal end 3019 of radial opening 3017, defined in tubular foreshaft's 301 wall, to point where target 900 abuts article 800 or its portion, and the step of anchoring article 800 or its portion to target 900 can then comprise: retracting either sheath 200 in a proximal direction beyond proximal end 3013 of tubular foreshaft 301, or retracting coupling rod 303 in a proximal direction beyond proximal end 3013 of tubular foreshaft 301; removing tab 3021 extending radially from pushrod's 302 distal end from radial opening 3017 defined in tubular foreshaft 301; and retracting pushrod 302 in a proximal direction beyond distal end 3013 of tubular foreshaft 301, causing strained proximal region 3012 of tubular foreshaft 301 to transition to unstrained position, hooking article 800 or its portion, thereby anchoring article 800 or its portion to target 900.

Additionally, or alternatively, in yet another exemplary implementation, of using bridging and anchoring construction 40, the step of translating bridging and anchoring construction 40 in distal direction relative to delivery catheter's 10, 10′ distal end 101 101′ comprises: using pushtube 402, translating bridging and anchoring construction 40 in a distal direction to the point where distal end 2502 of jacket 250 abuts target 900,; translating piercing tip 4010 in a distal direction, exposing piercing tip 4010; and piercing target 900 with piercing tip 4010, thereby causing each i^{th} lancing members 4010i to transition to unstrained position outside jacket 250 reversing each i^{th} lancing members'4010i direction, and thereby engaging target 900. Following engagement of target 900 as described, the step of translating bridging and anchoring construction 40 in a proximal direction comprises: using pushtube 402, translating bridging and anchoring construction 40 in a proximal direction, to a point where target 900 abuts article 800 or its portion. Similarly, albeit different, step of anchoring article 800 or its portion to target 900 comprises: retracting sheath 200 in a proximal direction beyond distal end 4020 of pushtube 402; and rotating pushtube 402 in a direction (clockwise or counter-clockwise) configured to cause pushtube 402 to translate proximally, causing strained proximal portion 4013 of foreshaft 401 to transition to unstrained position, once proximal end 4014 with pegs 4015, 4015′ exit distal end 4020 of pushtube 402, thereby forming a hook over article 800 or its portion, thereby anchoring article 800 or its portion to target 900.

Once article 800 is anchored to the target 900, the method further comprises, decoupling delivery catheter 10 from article 800 or its portion, translating sleeve 100 in a distal direction over the expander 110, transitioning expander 110 from its unstrained position to a strained position; and retracting delivery catheter 10.

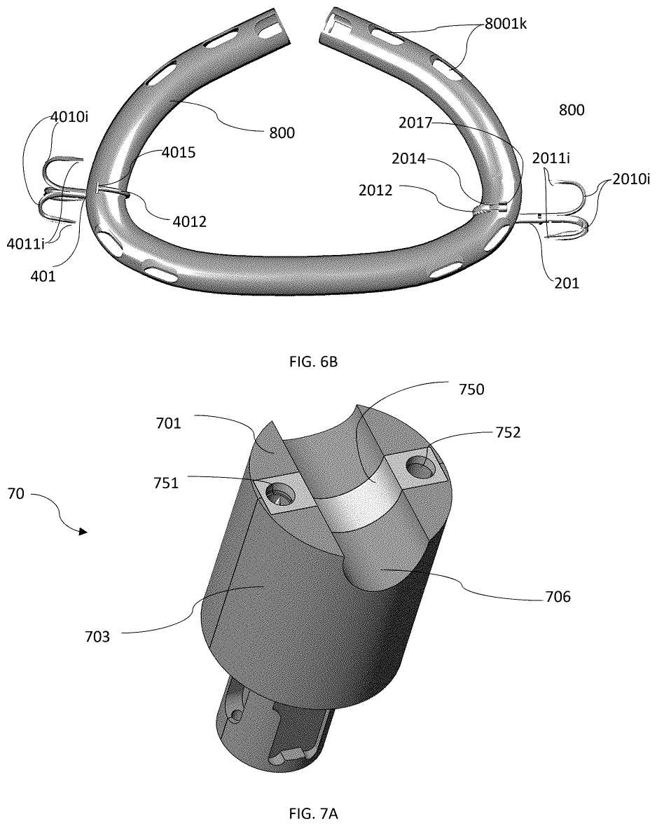

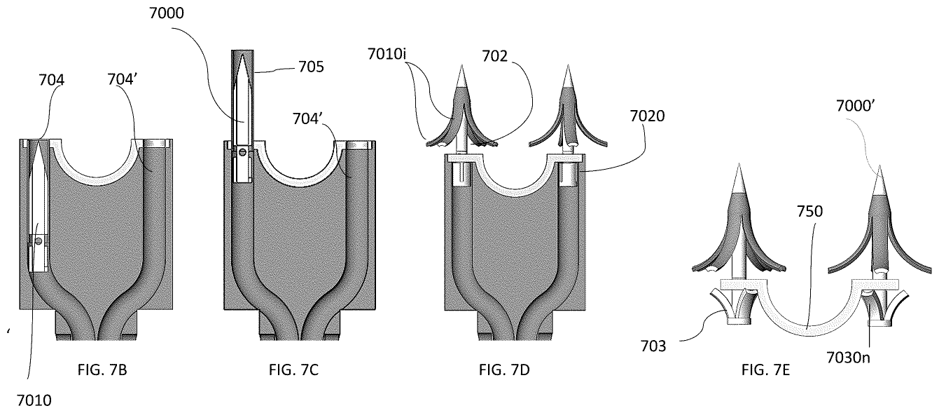

FIGS. 7A-8D, illustrate additional exemplary implementations of the system for bridging the gap between an article and a target. For Example, as illustrated in FIG. 7A, delivery catheter head 70, having distal end 701 and arcuate surface 706, accommodating omega-shaped clamp 750, defining pair of apertures 751, 752, sized to accommodate pair of bridging, engaging and anchoring constructs 7000, 7000′, each having piercing tip 7010, 7010′ and engagement base 703, operable to transition between strained and unstrained configuration within sheath 705. As illustrated, engaging the target on both sides of the article while abutting the delivery catheter 70 against the article to the point the article abuts the target, then distally translating lancing members 7010i beyond sheath 705, will initially cause lancing members 7010i to expand and further distal translation will transition anchoring base 703 spurs 7030n, to unstrain, anchoring clamp 750 over article 800.

Additionally, or alternatively, another exemplary implementation is illustrated in FIGS. 8A-8E, where ratcheting member 85 is operably coupled to cylindrical housing 83 of elongated delivery catheter 80 accommodating bridging, engaging and anchoring constructs 8000, having resilient laddered tail 88, configured upon translation of sheath 81 in a proximal direction, following engagement of the target, and distal translation of elongated delivery catheter 80 toward the target, to cause resilient tail to enter opening 8501 in ratcheting member 85, and engage protruding tab 8502, thereby using ratcheting member 85 as an anchor for the article, once ratcheting member 85 is decoupled from elongated delivery catheter 80.

While in the foregoing specification the methods, systems, sub systems (e.g., bridging and anchoring construction 20, 30, 40) use in bridging the gap between an article or a portion thereof and a target, engaging the target, and anchoring the article or its portion to the target described herein have been described in relation to certain exemplary implementations, and many details are set forth for purpose of illustration, it will be apparent to those skilled in the art that the disclosure of the alignment methods, implementable using the systems disclosed herein are susceptible to additional implementations and that certain of the details described in this specification and as are more fully delineated in the following claims can be varied considerably without departing from the basic principles disclosed herein.

What is claimed is:

1. An annuloplasty ring deployment system comprising:
an annuloplasty ring comprising a first end and a second end configured to be adjoined, the annuloplasty ring configured to be secured to a structural heart valve; and
a delivery catheter comprising a proximal end, distal end, and a bridging and anchoring construction configured to engage the annuloplasty ring at the distal end of the delivery catheter, the bridging and anchoring construction comprising a sheath, a pushtube configured to be disposed within the sheath, and an anchoring article configured to be disposed within the sheath and to interface with the pushtube such that distal advancement of the pushtube with respect to the sheath results in advancement of the anchoring article with respect to the sheath causing the anchoring article to at least partially exit the sheath and enter target tissue of the structural heart valve to secure the annuloplasty ring to the target tissue,
wherein a gap is defined between the annuloplasty ring when secured to the target tissue by the anchoring article and a second portion of the structural heart valve, and
wherein the bridging and anchoring construction is configured to move proximally to pull the annuloplasty ring towards the second portion of the structural heart valve, thereby reducing or eliminating the gap.

2. The annuloplasty ring deployment system of claim 1, wherein the anchoring article comprises a plurality of lancing members forming a piercing tip.

3. The annuloplasty ring deployment system of claim 2, wherein each lancing member of the plurality of lancing members has a sharpened tip.

4. The annuloplasty ring deployment system of claim 2, wherein each lancing member of the plurality of lancing members is configured to transition between a strained position when the anchoring article is within the sheath and an unstrained position when the anchoring article exits the sheath.

5. The annuloplasty ring deployment system of claim 4, wherein the plurality of lancing members are configured to form a three-pronged structure in the unstrained position.

6. The annuloplasty ring deployment system of claim 4, wherein the plurality of lancing members are configured to reverse direction to form the three-pronged structure.

7. The annuloplasty ring deployment system of claim 2, wherein the anchoring article comprises a tubular foreshaft coupled to the plurality of lancing members.

8. The annuloplasty ring deployment system of claim 1, wherein the anchoring article is configured to be releasably coupled with the pushtube such that the anchoring article is coupled to the pushtube while the anchoring article is positioned within the sheath and decoupled from the pushtube when the anchoring article exits the sheath.

9. The annuloplasty ring deployment system of claim 8, wherein the anchoring article is configured to be secured to the annuloplasty ring when decoupled from the pushtube.

10. The annuloplasty ring deployment system of claim 9, wherein the anchoring article is configured to form a hook over the annuloplasty ring when decoupled from the pushtube to secure the annuloplasty ring to the target tissue.

11. A method for deploying an annuloplasty ring, the method comprising:
delivering an annuloplasty ring coupled to a distal end of a delivery catheter to a structural heart valve using the delivery catheter, the delivery catheter comprising a proximal end, the distal end, and a bridging and anchoring construction comprising a sheath, a pushtube disposed within the sheath, and an anchoring article disposed within the sheath and in mechanical communication with the pushtube;
causing the pushtube of the bridging and anchoring to advance distally with respect to the sheath to cause distal advancement of the anchoring article with respect to the sheath causing the anchoring article to at least partially exit the sheath and enter target tissue of the structural heart valve to secure the annuloplasty ring to the target tissue; and
causing the bridging and anchoring construction to move proximally to pull the annuloplasty ring towards a second portion of the structural heart valve, thereby reducing or eliminating a gap defined between the annuloplasty ring when secured to the target tissue by the anchoring article and the second portion of the structural heart valve.

12. The method of claim 11, wherein the anchoring article comprises a plurality of lancing members forming a piercing tip.

13. The method of claim 12, wherein each lancing member of the plurality of lancing members has a sharpened tip.

14. The method of claim 12, wherein each lancing member of the plurality of lancing members is configured to transition between a strained position when the anchoring article is within the sheath and an unstrained position when the anchoring article exits the sheath.

15. The method of claim 14, wherein the plurality of lancing members are configured to form a three-pronged structure in the unstrained position.

16. The method of claim 14, wherein the plurality of lancing members are configured to reverse direction to form the three-pronged structure.

17. The method of claim 12, wherein the anchoring article comprises a tubular foreshaft coupled to the plurality of lancing members.

18. The method of claim 11, wherein the anchoring article is configured to be releasably coupled with the pushtube such that the anchoring article is coupled to the pushtube while the anchoring article is positioned within the sheath and decoupled from the pushtube when the anchoring article exits the sheath.

19. The method of claim 18, further comprising causing the anchoring article to fully exit the sheath and decouple from the pushtube.

20. The method of claim 19, wherein the anchoring article is configured to be secured to the an annuloplasty ring when decoupled from the pushtube.
